(19) United States
(12) Patent Application Publication
(10) Pub. No.: US 2007/0280411 A1
(43) Pub. Date: Dec. 6, 2007

(54) Method for the creation of panoramic tomographic images, and X-Ray image acquisition device

(75) Inventors: Johannes Ulrici, Heidelberg (DE); Ulrich Schulze-Ganzlin, Lorsch (DE)

Correspondence Address: DYKEMA GOSSETT PLLC, FRANKLIN SQUARE, THIRD FLOOR WEST, 1300 I STREET, NW, WASHINGTON, DC, 20005, US

(73) Assignee: Sirona Dental Systems GmbH

(21) Appl. No.: 11/783,106

(22) Filed: Apr. 5, 2007

(30) Foreign Application Priority Data
Apr. 6, 2006 (DE) 10 2006 016 572.1

(51) Int. Cl. A61B 6/00

(52) U.S. Cl. 378/25

(57) ABSTRACT

The invention relates to a method for the creation of a panoramic tomographic image of an object (10) by means of X-rays, in which a digital X-ray-sensitive image detector (14) is moved relatively to the object to be X-rayed (10) and image data of the object (10), for a first layer (15) of the object (10) are summated to a first storage area (21.1), wherein the summation is carried out after a predefined first time interval (Δt₁) with a predefined first line offset (Δs₁). Image data for a second layer (16) of the object (10) are summated to a second storage area (21.2), which summation is performed after a predefined second time interval (Δt₂) with a predefined second line offset (Δs₂).

The invention further relates to a digital X-ray image acquisition device (1) for the creation of panoramic tomographic images of an object (10), comprising an X-ray-sensitive image detector (14), a first and second storage area (21.1, 21.2) for storing data, and a first and second linker (20.1, 20.2) for linking image data.

The invention relates to a method for the production of a panoramic tomographic image of an object by means of X-rays, in which a digital X-ray-sensitive image detector is moved relatively to the object being tomographed, and image data for a first layer of the object is summated and appended to a first memory, said summation being undertaken after a predetermined first time interval with a predetermined first line offset.

The invention also relates to a digital X-ray image acquisition device for panoramic tomography of an object, which contains an X-ray-sensitive image detector, a first memory for storing data and a linker for assigning image data.

### DESCRIPTION OF THE RELATED ART

EP 0 279 293 discloses a dental X-ray diagnostic device for panoramic tomography of a patient's jaw, which device contains an A/D converter connected to a detector, an image memory and a data processing device, which computes an overall image from the signals supplied by the detector system during a tomographic operation.

It is also known from the prior art to use CCDs as sensors for tomography, said CCDs being operated in the TDI mode. The rate of displacement of the signal charges on the CCD is adapted to the relative speed of the CCD relative to the object being tomographed. In this way it is possible to create a sharp image of a certain layer of the object being tomographed.

With the procedures known in the prior art it is only possible to record a single sharp layer per revolution of the X-ray device. However, this is a disadvantage for several reasons. On the one hand, in the case of dental panoramic tomography, the sharp layer is not always exactly in the arch of the jaw since every jaw is individually shaped. On the other hand, it is possible that a pathological site that must be identified for planning treatment or for making a diagnosis is not sharply imaged because it may lie next to the sharp layer, for example. Moreover, it is sometimes desirable to prepare several tomographic images of adjacent planes of a region in order to obtain a tomographic overview of the region, for example.

All of these requirements necessitate one or more tomographic images, which, on the one hand, are time-consuming, and on the other hand, unnecessarily increase the radiation burden on the patient.

It is thus an object of the invention to provide a method and an X-ray device with which it is possible to simultaneously tomograph several sharp layers lying side by side.

### SUMMARY OF THE INVENTION

This object is achieved by the invention with a method for panoramic tomography as defined in claim 1 and a digital X-ray imaging device as defined in subclaim 10.

The method for producing panoramic tomographic images of an object by means of X-rays, in which a digital X-ray-sensitive image detector is provided whose pixels are arranged in a two-dimensional line/column pattern envisions that the image detector be moved relatively to the object being tomographed at a predefined velocity to record image data of the object, the image data for a first layer being read from the image detector at a predetermined read frequency, and, after each readout of the image detector, summated in a first storage area and appended to an associated memory content present in the first storage area, said summation being performed after a set first time interval with a set first line offset, said first time interval also being a whole-number multiple of the reciprocal of the first read frequency.

Image data for a second layer are read with a second read frequency from the image detector, and after each reading of the image detector summated in a second storage area to an associated memory content present in the second storage area. The summation is performed after a predetermined second time interval with a predetermined second line offset, the second time interval being a whole-number multiple of the reciprocal of the second read frequency.

The memory content present in the storage areas can be an image obtained from image information previously read out from the image detector, a summated image of several such image data sets, or the memory may be empty.

This method makes it possible to simultaneously record two layers of the object being imaged, in which case the layers may be in any position relative to each other.

It is especially advantageous if image data for other layers are read out from the image detector with other predetermined read frequencies and, after each readout of the image detector, are summated in other storage areas and appended to a respective associated memory content present in the other storage area. The summation is performed after predetermined other time intervals with predetermined other line offsets, in which case the other time intervals are each a whole number multiple of the reciprocal of the other read frequencies respectively. In this way it is possible to tomograph several layers simultaneously and obtain a tomographic image of, say, a jaw.

The memory contents of each storage area can advantageously be read out and summated with the newly recorded image data with the given line offset in each case, and the summated data saved in the respective storage area. The memory contents may be summated data from previous summations. The image data may also be stored and summated in digital form.

Instead of a summating unit other linkers may also be provided, e.g. subtractors. This only changes the specific design, but not the underlying principle of the invention.

The read frequencies are advantageously equal to a common read frequency. Such a method can be carried out at lower cost.

The respective time intervals are advantageously different from one another. This permits imaging of different layers.

The whole-number multiples are advantageously time-dependent. This permits variation of the relative position of the layers during the tomographic operation. For instance it is conceivable that in a region of, say, a front tooth, it becomes necessary to bring the layers closer to one another than in another region, say, a molar region.

It is especially advantageous if the data present in the storage areas are written into another memory. This other memory serves to store the finished panoramic tomograms of the layers in question and can be located externally of the device used for processing and can, for example, be in the form of a computer hard disk. The other memory forms the basis for retrieval of the image information for diagnostic purposes.

It is especially advantageous if the predefined rate and/or the read frequencies are time-dependent. This increases flexibility in adapting the relative positions of the sharp layers.

Instead of time dependence, location dependence of the location of the X-ray emitter and the image detector may be of advantage if a predetermined trajectory is being followed. The two dependencies are in a distinct relationship to each other due to a known equation of motion of the X-ray apparatus and are thus interchangeable.

The image detector is advantageously reset at a given frequency. The given frequency may be time-dependent.

The digital X-ray imaging device of the invention for panoramic tomography of an object includes an X-ray-sensitive image detector, whose pixels are arranged in a two-dimensional line pattern, a first memory for storing data and a linker for linking image data which cooperates with a first storage area and the image detector. In addition, another storage area and a clock unit are present, the clock unit offering several clock frequencies that control reading and writing of image information.

Such an X-ray tomography device permits the recording of several sharp layers in a single pass.

The image detector is advantageously designed as a CMOS image detector. CMOS image detectors permit a higher degree of integration of subassemblies on the image detector than CCD sensors and can also be manufactured at lower cost.

Advantageously, one or more additional storage areas are present for the storage of data that cooperate with the linker, while the clock unit offers one or more additional clock frequencies.

This makes it possible to tomograph further sharp layers.

It is especially advantageous if the storage areas are logical regions of a common memory. This lowers the production costs.

Advantageously, the time interval between two line offsets is a whole-number multiple of the reciprocal of the respective read frequency.

The linker advantageously carries out two linkages, each with a pre-assigned line offset. In this way it becomes possible to dispense with other means of producing a line offset.

It is especially advantageous if the respective line offset between two time intervals is a whole-number multiple of the reciprocal of the respective read frequency. This achieves the linkage of complete images with each other.

The read frequencies are advantageously equal to a common read frequency. This reduces the equipment costs without imposing excessive limitation on the functionality of the X-ray imaging device.

The time intervals at which a line offset is performed differ from one another for different memories and linkers. In the event of identical read frequencies, therefore, the tomography of different layers will be possible.

The memory is advantageously designed as an analog memory and the linker as an analog linker. Such linkers and memories are very fast and can be installed on the board of the image detector in a space-saving manner. This reduces signal deterioration due to long conduction paths and intermediate digitization of the individual tomograms which, in sum total, leads to greater image deviations than does digitization of the finished summated image. In addition, the number of digitizations can be reduced, which means that only slower and/or fewer AD converters will be necessary.

As an alternative, it is possible to design the memory as a digital memory and configure the linker such that it can read the digital memory contents from the storage areas. In this way corrections can be made at an early stage on the partial images.

Advantageously, another memory is provided that cooperates with the memory and serves for permanent storage of data. This allows for the storage of X-ray images for diagnostic purposes.

### BRIEF DESCRIPTION OF THE DRAWINGS

The method of the invention and the X-ray tomographic device are explained in more detail with reference to the figures, in which:

FIG. 1 shows an X-ray tomographic device,

FIG. 2 is a basic diagram illustrating the principle of tomographing different layers,

FIG. 3 shows the basic imaging-side structure of the X-ray image acquisition device in a first embodiment,

FIG. 4 shows the basic imaging side structure of the X-ray image acquisition device in a second embodiment, and

FIG. 5 shows a basic diagram illustrating the method of the invention.

### DETAILED DESCRIPTION OF THE INVENTION

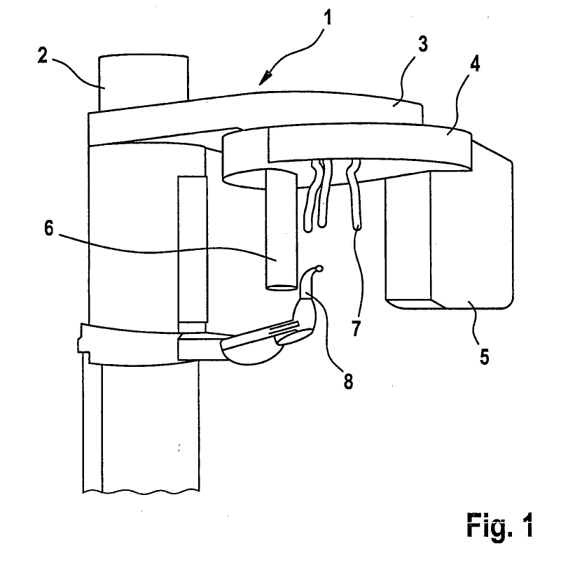

FIG. 1 shows an X-ray device 1 according to the invention. On a supporting column 2 there is provided a boom 3 on which a carrier 4 is mounted for rotation. On the carrier 4 an X-ray emitter 5 and an image detector 6 are disposed diametrically opposed to a holding device 7 for a patient (not shown).

The patient is positioned by the holding device 7 and a mouthpiece 8 such that the patient's head is kept stationary with respect to the supporting column 7. The carrier 4 then revolves along a predetermined path around the patient's head.

In the known TDI method based on the use of CCD sensors, the image information detected thus far is moved contrary to the direction of motion of the image detector relatively to the object being tomographed such that the image data of a point being imaged always expose the cell to which the image data of the same point being imaged was moved from the previous tomograph. The image data are therefore moved contrary to the imaging direction in such a way that they are located in a pixel of the image detector that lies on a straight line passing from the X-ray source to the image pixel through the point being imaged. The rate of displacement of the image data on the sensor must therefore be adapted to the position of the sharp layer.

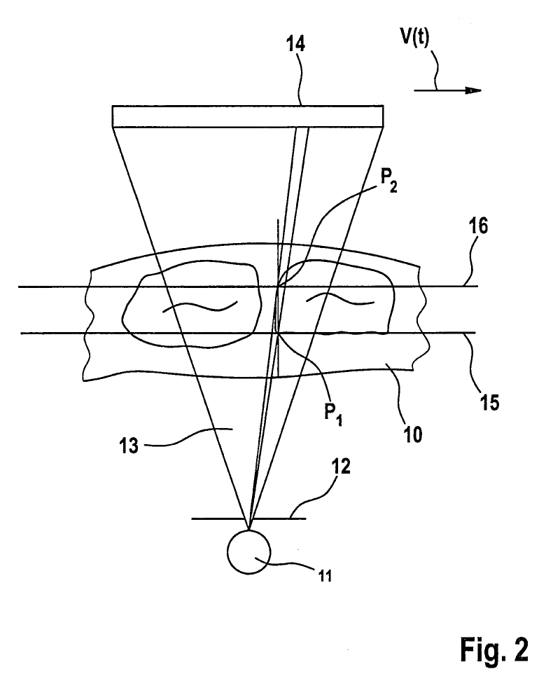

FIG. 2 shows a basic diagram for illustration of the principle for the production of a layer based on a jaw section 10 of the patient. For the sake of clarity, the diagram is not drawn to scale.

The X-ray source 11 emits a fan beam 13 through a primary aperture 12, to penetrate the jaw segment 10 and impinges on the image detector 13, where it is recorded. Also shown are two layers of the jaw segment 10 being tomographed, a first layer 15 and a second layer 16.

In the small region of the jaw segment 10 being imaged shown in FIG. 2, the movement of the recording unit consisting of X-ray source 11, primary aperture 12 and image detector 14 can be approximated as linear relative to the jaw segment 10 so that the X-ray unit moves relative to the jaw segment 10 at a velocity v(t) parallel to the layers 15 and 16 being imaged.

The closer a layer to be imaged is to the image detector 14, the more slowly its image moves, according to the principles of intercept theorems, on the image detector 14 when in relative motion across the image detector 14. A point P₁ on the first layer 15, here relating to the boundary between a tooth and a jaw, travels at a greater speed across the image detector than another point P₂ on the second layer 16, which is on the same level.

For geometric reasons, therefore, the necessary displacement speed of the image data on the image detector 14 for the sharp layer 16 is lower than the corresponding displacement speed for the sharp layer 15.

The desired spacing between the sharp layers to be imaged varies during image acquisition. In a front tooth region, for example, it could be necessary for the sharp layers to be closer together than in a molar region.

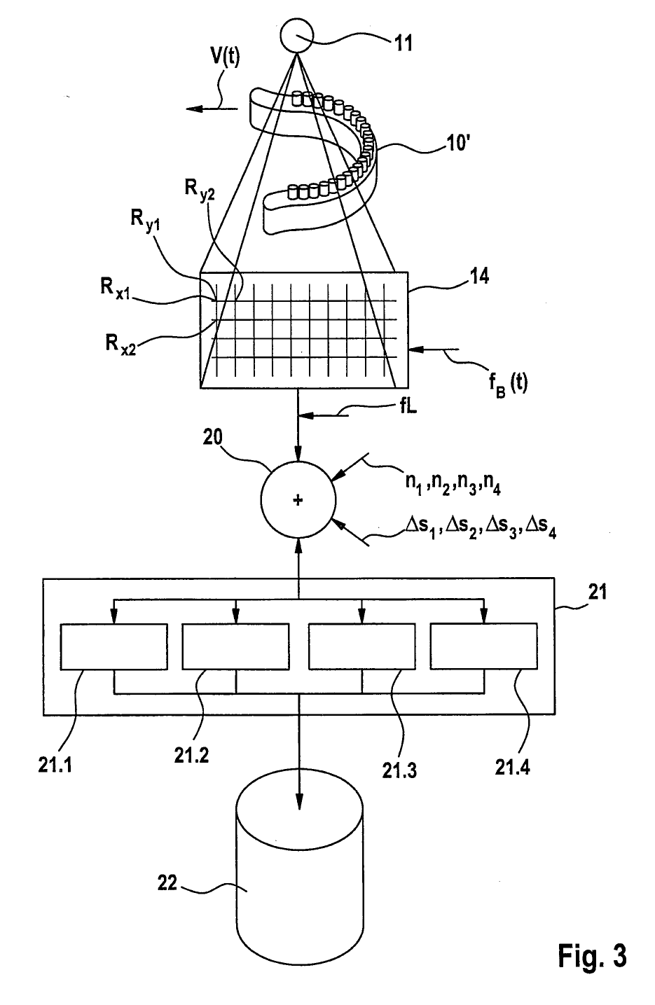

FIG. 3 shows the image-acquisition side part of the X-ray imaging device 1 in a first embodiment. The jaw 10′ irradiated by the X-ray source 11 is imaged on the image detector 14.

The image detector 14 is configured as a CMOS sensor 14. The CMOS sensor 14 can be read out without deleting the charges in the pixels. This makes it possible to read the CMOS sensor 14 as often as desired. Deletion of pixels is independent of the readout.

The pixels of the CMOS sensor 14 are arranged in a two-dimensional line pattern on columns Rₓ₁, Rₓ₂ and lines R_{y1}, R_{y2}, etc. . . . The CMOS sensor 14 registers image data on the jaw 10′ at a frequency f_{B}(t), which means that the charge content of the pixels of the CMOS sensor 14 is reset after each clock pulse 1/f_{B}(4t)

A summating unit 20 reads image data from the CMOS sensor 14 at a read frequency f_{L}(t) and sums it up in a memory 21. For this purpose the image data read out of the CMOS sensor 14 are summated in pixel form on the memory content present in the memory 21. The memory content present in the storage areas can be an image of image information previously read out from the image detector, a summated image produced from several such image data, or the memory can be empty, if the memory has been emptied in the preceding clock cycle as described below.

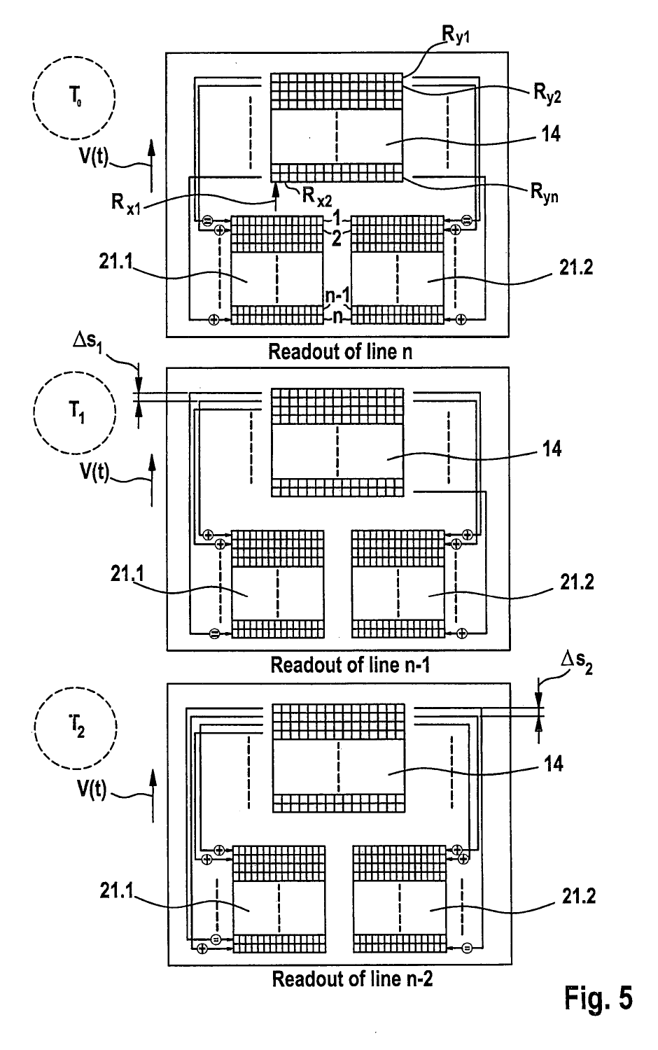

As an example, the memory 21 is divided up into four logical storage areas 21.1, 21.2, 21.3, 21.4, into which the summating unit 20 summates data by the method described in more detail with reference to FIG. 5. The summated image data are then passed on to a second storage area 22 in which they are saved/filed and held ready for evaluation.

Depending on the physical configuration of the sensor, it may be necessary to effect summation by reading out the respective memory contents of the storage areas 21.1, 21.2, 21.3, 21.4, and to write them back to the respective storage area 21.1, 21.2, 21.3, 21.4, following summation.

The summating unit 20 is basically controlled during summation by other parameters n₁(t), n₂(t), n₃(t), n₄(t), Δs₁, Δs₂, Δs₃, Δs₄, whose function is explained in more detail with reference to FIG. 5.

It is still possible to provide an analog amplifier between the CMOS sensor 14 and the summating unit 20.

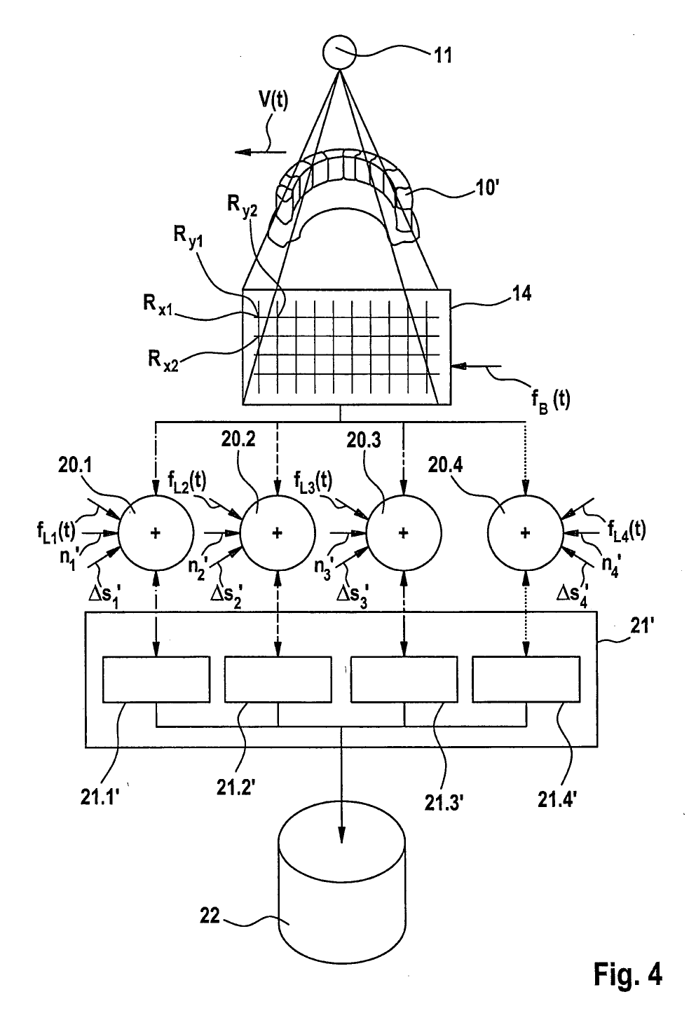

FIG. 4 shows the image acquisition-side structure of the radiographic device 1 in a second embodiment. Unlike the embodiment shown in FIG. 3, here a plurality of summating units 20.1, 20.2, 20.3, and 20.4 is provided. Each of these summating units 20.1, 20.2, 20.3 und 20.4 operates with its own read frequency f_{L1}(t), f_{L2}(t), f_{L3}(t) and f_{L4}(t). To each summating unit 20.1, 20.2, 20.3 und 20.4 there is assigned a line offset Δs₁′, Δs₂′, Δs₃′ und Δs₄′ as well as, for each, an whole number n₁′(t), n₂′(t), n₃′(t), and n₄′(t) is preset, to control the memory logic. Each of these summating units 20.1, 20.2, 20.3 und 20.4 is associated with a storage area 21.1′, 21.2′, 21.3′ and 21.4′ of a memory 21′.

The storage and summating process is explained in more detail with reference to FIG. 5.

The velocity v(t), the imaging frequency f_{B}(t), the whole numbers n₁(t), n₂(t), n₃(t), n₄(t), n₁′(t), n₂′(t), n₃′(t), n₄′(t), and the read frequencies f_{L1}(t), f_{L2}(t), f_{L3}(t), and f_{L4}(t) are time dependent, said time dependence being a function of the region of the jaw 10′ to be imaged. It is therefore also possible to represent the aforementioned magnitudes as a function of the location of the X-ray apparatus. The speed of revolution of the X-ray emitter 5 and of the image detector 6 around the jaw 10′ of the patient is dependent on the position of the X-ray emitter and the image detector 6 relative to the jaw.

The summating units 20.1, 20.2, 20.3, and 20.4 as well as the memory 21′ are designed as analog structures. In the storage areas 21.1′, 21.2′, 21.3′, and 21.4′ the signals of the image detector 14 are summated in the analog mode. This has the advantage that the summating units 20.1, 20.2, 20.3, and 20.4 and the memory 21′ can be put on the CMOS chip without there being any necessity for extremely fast digitization. The analog structures are space-saving, the short signal paths and direct processing of the image signals without prior digitization improve the precision and the signal-to-noise ratio, and the analog structures are, in addition, sufficiently fast.

Instead of summating, other linkages may be undertaken, e.g., subtraction of two consecutive recorded images and subsequent addition of the differential images resulting from the subtraction. This is advantageous whenever the CMOS sensor 14 is reset with a frequency f_{B}(t), which is lower than the accordingly read frequency f_{L1}(t), f_{L2}(t), f_{L3}(t), and f_{L4}(t). By forming the difference between two successively readout memory contents it is possible to ascertain the newly acquired information content.

It is still possible to provide an analog amplifier between the CMOS sensor 14 and the linker 20.

FIG. 5 illustrates the method of acquiring TDI images by means of the CMOS sensor 14. The principle employed for the simultaneous production of two layers by means of the storage areas 21.1 and 21.2 is illustrated. Unlike FIG. 3, the sensor is shown in a rotated position so that v(t) points upwardly.
Three times are shown, T₀, T₁ und T₂, where: T₁=T₀+n₁(t)/f_{L1}(t);
T₂=T₀+2×n₁(t)/f_{L1}(t), and
T₂=T₀+n₂(t)/f_{L2}(t) where n₂(t)=2×n₁(t).

Time T₀ represents the time when the memory areas 21.1 and 21.2 are just being written. In this case the image information which is present in the CMOS sensor 14 in line R_{y1} is written into line 1 of the two storage areas 21.1 and 21.2.

At time T₁, a line offset Δs₁ in memory area 21.1 of one line is effected. The first line R_{y1} is written into the last written line of the storage area 21.1, which had been deleted at the previous clock pulse, and the second line R_{y2} is appended to the contents of the first line 1 of the first storage area 21.1.

The image information is summated and appended to the second storage area 21.2 in the same way as at time T₀.

After each summation cycle individual lines of storage areas 21.1 and 21.2 are read out and sent to memory 22, which stores the data.

At the time T₂, another line offset is effected in the storage area 21.1, so that the line R_{y1} is added to line n−1 of storage area 21.1. The line R_{y2} is appended to the line n of the storage area 21.2.

In the storage area 21.2, after a line offset Δs₂ of one line, summation to line 2 is effected, as was carried out at time T₁ in storage area 21.1.

Together with the readout of the lines in the storage areas 21.1 and 21.2 the lines are reset. At the time T₀ the line n is read out and reset. The readout in the next clock cycle is then performed in the previous line, here therefore n−1. What this achieves is that summation is performed just as often into each line before the readout. Then at time T₂ the line n−2 is read out.

It is possible to specify other parameterizations leading to the same result. For example, the line offset Δs can be represented as a function of time Δs(t) so that the line offset varies throughout the cycles. In the following case it would then be true that:
Δs₁(Tᵢ)=1; i=1, 2, 3, . . . ,
Δs₂(T_{i′})=1; i′=2, 4, 6, . . . .

However, this does not depart from the basic scope of the present invention.

### LIST OF REFERENCE NUMERALS

1 X-ray apparatus
2 Supporting column
3 Boom
4 Rotatable carrier
5 X-ray emitter
6 Image detector
7 Holding device
8 Mouthpiece
10 Jaw segment
11 X-ray source
12 Primary aperture
13 Fan beam
14 Image detector
15 First layer
16 Second layer
20 Summating unit
20.1 First summating unit
20.2 Second summating unit
20.3 Third summating unit
20.4 Fourth summating unit
21 Memory
21′ Memory
21.1 First storage area
21.2 Second storage area
21.3 Third storage area
21.4 Fourth storage area
21.1′ First storage area
21.2′ Second storage area
21.3′ Third storage area
21.4′ Fourth storage area
22 Memory
Δs₁ First line offset
Δs₂ Second line offset
Δs₃ Third line offset
Δs₄ Fourth line offset
Δs₁′ First line offset
Δs₂′ Second line offset
Δs₃′ Third line offset
Δs₄′ Fourth line offset
n₁(t) First whole number
n₂(t) Second whole number
n₃(t) Third whole number
n₄(t) Fourth whole number
n₁(t) First whole number
n₂′(t) Second whole number
n₃′(t) Third whole number
n₄′(t) Fourth whole number
Δt₁ First time interval
Δt₂ Second time interval
Δt₃ Third time interval
Δt₄ Fourth time interval
Δt₁′First time interval
Δt₂′ Second time interval
Δt₃′ Third time interval
Δt₄′ Fourth time interval
T₀, T₁, T₂ Points of time
P₁ First point
P₂ Second point
Rₓ₁ First column
Rₓ₂ Second column
R_{y1} First line
R_{y2} Second line
f_{B}(t) Imaging frequency
f_{L}(t) Read frequency
f_{L1}(t) First read frequency
f_{L2}(t) Second read frequency
f_{L3}(t) Third read frequency
f_{L4}(t) Fourth read frequency

1. A method for the creation of panoramic tomographic images of an object (10, 10′) by means of X-rays (13), in which a digital X-ray-sensitive image detector (14), whose pixels are arranged in a two-dimensional line pattern (Rₓ₁, Rₓ₂, R_{y1}, R_{y2}), is moved at a fixed speed (V(t)) relatively to the object to be X-rayed (10, 10′) to record image information of the object (10, 10′), wherein the image data for a first layer (15) are read from the image detector (14) at a predefined first read frequency (f_{L}(t), f_{L1}(t)) and, following each readout of the image detector (14), are summated in a first storage area (21.1, 21.1′) and appended to associated memory contents present in the first storage area (21.1, 21.1′), wherein the summation is carried out after a predefined first time interval (Δt₁, Δt₁′) with a predefined first line offset (Δs₁, Δs₁′), and wherein the first time interval (Δt₁, Δt₁′) is an integral multiple (n₁, n₁′) of the reciprocal of the first read frequency (f_{L}(t), f_{L1}(t)), characterized in that image data for a second layer (16) are read from the image detector (14) at a predefined second read frequency (f_{L}(t), f_{L2}(t)) and, following each readout of said image detector (14) are summated in a second storage area (21.2, 21.2′) and appended to associated memory contents present in said second storage area (21.2, 21.2′), which summation is performed after a predefined second time interval (Δt₂, Δt₂′) with a predefined second line offset (Δs₂, Δs₂′), the second time interval (Δt₂, Δt₂′) being an whole-number multiple (n₂, n₂′) of the reciprocal of the second read frequency (f_{L}(t), f_{L2}(t)).

2. A method as defined in claim 1, characterized in that image data for further layers are read from the image detector (14) at other predetermined read frequencies (f_{L}(t), f_{L3}(t), f_{L4}(t)) and, after each readout from said image detector (14), are summated in further storage areas (21.3, 21.3′, 21.4, 21.4′) and appended to associated memory contents present in respective further storage areas (21.3, 21.3′, 21.4, 21.4′), the summation being performed after other predefined time intervals (Δt₃, Δt₃′, Δt₄, Δt₄′) with other pre-defined line offsets (Δs₃, Δs₃′, Δs₄, Δs₄′), the other time intervals (Δt₃, Δt₃′, Δt₄, Δt₄′) each being a whole-number multiple (n₃, n₃′, n₄, n₄′) of the reciprocal of the said other read frequencies (f_{L}(t), f_{L3}(t), f_{L4}(t)).

3. A method as defined in claim 1 or claim 2, characterized in that the memory contents are read from each of said storage areas (21.1, 21.1′, 21.2, 21.2′, 21.3, 21.3′, 21.4, 21.4′), are summated with the respective image information with the given line offset (Δs₁, Δs₁′, Δs₂, Δs₂′, Δs₃, Δs₃′, Δs₄, Δs₄′) and the summated data are written back to the respective storage area (14), while the image data from the respective storage area (14) can be summated data from previous summations.

4. A method as defined in any one of claims 1 to 3, characterized in that said read frequencies (f_{L1}(t), f_{L2}(t), f_{L3}(t), f_{L4}(t)) are equal to a common read frequency (f_{L}(t)).

5. A method as defined in any one of claims 1 to 4, characterized in that the respective time intervals (Δt₁, Δt₂, Δt₃, Δt₄) differ from each other.

6. A method as defined in any one of claims 1 to 5, characterized in that the whole-number multiples (n₁, n₁′, n₂, n₂′, n₃, n₃′, n₄, n₄′) are time-dependent (n₁(t), n₁′(t), n₂(t), n₂′(t), n₃(t), n₃′(t), n₄(t), n₄′(t)).

7. A method as defined in any one of claims 1 to 6, characterized in that the data present in said storage areas (14) are written to another memory (22).

8. A method as defined in any one of claims 1 to 7, characterized in that the fixed speed (v(t)) and/or the read frequencies (f_{L}(t), f_{L1}(t), f_{L2}(t), f_{L3}(t), f_{L4}(t)) are time-dependent.

9. A method as defined in any one of claims 1 to 8, characterized in that the image detector (14) is reset periodically.

10. A digital X-ray image acquisition device (1) for the creation of panoramic tomographic images of an object (10, 10′), comprising an X-ray-sensitive image detector (14), whose pixels are arranged in a two-dimensional line pattern (Rₓ₁, Rₓ₂, R_{y1}, R_{y2}), a first storage area (21.1, 21.1′) for storing data, a linker (20, 20.1, 20.2, 20.3, 20.4) for linking image data, cooperating with the first storage area (21.1, 21.1′) and the image detector (14), characterized in that another storage area (21.2, 21.2′) is present and that a clock unit is present, which provides a plurality of clock frequencies (f_{L}(t), f_{L1}(t), f_{L2}(t)) are provided for the control of readout and writing of said image information.

11. An X-ray image acquisition device (1) as defined in claim 10, characterized in that said image detector (14) is a CMOS image detector.

12. An X-ray image acquisition device (1) as defined in claim 10 or claim 11, characterized in that one or more other storage areas (21.3, 21.3′, 21.4, 21.4′) are provided for the storage of data and cooperate with said linker (20, 20.1, 20.2, 20.3, 20.4), and the clock unit provides one or more further clock frequencies (f_{L3}(t), f_{L4}(t)).

13. An X-ray image acquisition device (1) as defined in any one of claims 10 to 12, characterized in that said storage areas (21.1, 21.1′, 21.2, 21.2′, 21.3, 21.3′, 21.4, 21.4′) are logical regions of a memory (21).

14. An X-ray image acquisition device (1) as defined in any one of claims 10 to 13, characterized in that each linker (20, 20.1, 20.2, 20.3, 20.4) creates two links with a predetermined line offset (Δs₁, Δs₁′, Δs₂, Δs₂′, Δs₃, Δs₃′, Δs₄, Δs₄′).

15. An X-ray image acquisition device (1) as defined in claim 14, characterized in that the respective time interval (Δt₁, Δt₂, Δt₃, Δt₄, Δt₁′, Δt₂′, Δt₃′, Δt₄′) between two line offsets (Δs₁, Δs₁′, Δs₂, Δs₂′, Δs₃, Δs₃′, Δs₄, Δs₄′) is in each case an whole-number multiple (n₁(t), n₁′(t), n₂(t), n₂′(t), n₃(t), n₃′(t), n₄(t), n₄′(t)) of the reciprocal of the respective read frequency (f_{L}(t), f_{L1}(t), f_{L2}(t), f_{L3}(t), f_{L4}(t)).

16. An X-ray image acquisition device (1) as defined in any one of claims 8 to 15, characterized in that the read frequencies (f_{L1}(t), f_{L2}(t), f_{L3}(t), f_{L4}(t)) are equal to a common read frequency (f_{L}(t)).

17. An X-ray image acquisition device (1) as defined in any one of claims 14 to 16, characterized in that the time intervals (Δt₁, Δt₂, Δt₃, Δt₄, Δt₁′, Δt₂′, Δt₃′, Δt₄′) differ from each other.

18. An X-ray image acquisition device (1) as defined in any one of claims 10 to 17, characterized in that said memory (21) is designed as an analog memory and the linker (20, 20.1, 20.2, 20.3, 20.4) is designed as an analog linker (20, 20.1, 20.2, 20.3, 20.4).

19. An X-ray system as defined in any one of claims 10 to 17, characterized in that said memory (21) is designed as a digital memory and that the linker (20, 20.1, 20.2, 20.3, 20.4) can read memory contents from the storage areas (21.1, 21.2, 21.3, 21.4).

20. An X-ray image acquisition device (1) as defined in any one of claims 10 to 19, characterized in that a memory (22) for permanent storage of data is provided which cooperates with said memory (21).
